(19) United States
(12) Patent Application Publication
(10) Pub. No.: US 2024/0185991 A1
(43) Pub. Date: Jun. 6, 2024

(54) SYSTEM AND METHOD FOR HUMANIZING THE ONBOARDING BUDDY RECOMMENDATION PROCESS

(71) Applicant: SAP SE, Walldorf (DE)

(72) Inventors: Bharath Ganesh, Bangalore (IN); Vasanthi Valliammai V, Bangalore (IN); Neha Vishwanath, Bangalore (IN); Mohd Tahir, Bangalore (IN); Ajith Jose, Bangalore (IN)

(21) Appl. No.: 18/074,755

(22) Filed: Dec. 5, 2022

(51) Int. Cl. G16H 40/20

(52) U.S. Cl. CPC G16H 40/20

(57) ABSTRACT

The present disclosure involves systems, software, and computer implemented methods for humanizing the onboarding buddy recommendation process. One example method includes obtaining, at an employee management software system, a first psychometric profile of a first person. A plurality of psychometric profiles of a plurality of persons from a database are accessed by the employee management software system from a database. The first psychometric profile and the plurality of psychometric profiles are processed by the employee management software system through a self-learning machine learning model to generate a list of persons. A second person in the list is assigning, within the employee management software system, to have a matching relationship with the first person.

### TECHNICAL FIELD

The present disclosure relates to computer-implemented methods, software, and systems for humanizing the onboarding buddy recommendation process in the human resources (HR) domain.

### BACKGROUND

The onboarding buddy recommendation process provides a mechanism to assign, within an organization, a current employee as a buddy to a new employee. The assigned buddy may help with smooth onboarding of the new employee, and act as a first point of contact of the organization for the new employee. Current onboarding solutions do not automatically, and without user input, consider human factors (such as personality traits) while assigning or recommending a buddy.

### SUMMARY

The present disclosure involves systems, software, and computer implemented methods for humanizing the onboarding buddy recommendation process. An example method includes obtaining, at an employee management software system, a first psychometric profile of a first person. A plurality of psychometric profiles of a plurality of persons are accessed from a database by the employee management software system. The employee management software system can then process the first psychometric profile and the plurality of psychometric profiles through a self-learning machine learning (ML) model to generate a list of persons. Within the employee management software system, a second person in the list can be assigned to have a matching relationship with the first person.

A first feature, combinable with any of the following features, wherein processing the first psychometric profile and the plurality of psychometric profiles through the self-learning machine learning model comprises retrieving a first feature vector of the first person from the first psychometric profile, retrieving a plurality of feature vectors of the plurality of persons from the plurality of psychometric profiles, and comparing the first feature vector to the plurality of feature vectors.

A second feature, combinable with any of the previous or following features, wherein, before processing the first psychometric profile and the plurality of psychometric profiles, the self-learning machine learning model is trained with historical data.

A third feature, combinable with any of the previous or following features, wherein the list of persons are derived from the plurality of persons, each person in the list is associated with a predicted efficiency of a matching relationship with the first person, and the second person has a relatively highest predicted efficiency among the list of persons.

A fourth feature, combinable with any of the previous or following features, wherein the first psychometric profile of the first person includes a score quantifying a core set of personality traits of the first person.

A fifth feature, combinable with any of the previous or following features, wherein the core set of personality traits includes openness to experience, conscientiousness, extraversion, agreeableness, and neuroticism.

A sixth feature, combinable with any of the previous or following features, wherein the core set of personality traits is obtained through a questionnaire or through analyzing one or more social media accounts of the first person.

A seventh feature, combinable with any of the previous or following features, wherein the first person is a new employee of an organization, the plurality of persons are current employees of the organization, and the employee management software system includes an onboarding system platform of the organization.

An eighth feature, combinable with any of the previous or following features, further comprising storing the first psychometric profile in the database.

A ninth feature, combinable with any of the previous or following features, wherein the matching relationship includes a buddy relationship or a mentorship.

While generally described as computer-implemented software embodied on tangible media that processes and transforms the respective data, some or all of the aspects may be computer-implemented methods or further included in respective systems or other devices for performing this described functionality. The details of these and other aspects and embodiments of the present disclosure are set forth in the accompanying drawings and the description below. Other features, objects, and advantages of the disclosure will be apparent from the description and drawings, and from the claims.

### DESCRIPTION OF DRAWINGS

FIG. 1 is a block diagram illustrating an example system for humanizing the onboarding buddy recommendation process.

FIG. 2 is a flow diagram of an example process for predicting and assigning an onboarding buddy.

FIG. 3 is a flowchart of an example method for humanizing the onboarding buddy recommendation process.

### DETAILED DESCRIPTION

The following detailed description describes humanizing the onboarding buddy recommendation process. Various modifications to the disclosed implementations will be readily apparent to those skilled in the art, and the general principles defined herein may be applied to other implementations and applications without departing from scope of the disclosure. Thus, the present disclosure is not intended to be limited to the described or illustrated implementations, but is to be accorded the widest scope consistent with the principles and features disclosed herein.

The onboarding buddy recommendation process can be used to assign or recommend a current employee as a buddy to a new employee in an organization. The assigned buddy acts as a first point of contact of the organization for the new employee, and can help with smooth onboarding of the new employee. For example, the assigned buddy can explain processes, facilities, policies, and teams of the organization to the new employee. An effective buddy (e.g., a buddy who gets along well with the new employee, or whose demeanor and interactions matches or complements the new employee's own) can improve new employee's onboarding experience, work efficiency, and connections to the organization. In existing onboarding solutions, a manager or an HR personnel manually assigns or recommends a current employee as the new employee's buddy. However, human factors (such as personality traits) are not automatically considered while assigning or recommending the buddy by the system. In other words, no initial technical analysis of existing and evaluated personal traits is used in the assignment of the buddy. Not automatically considering human factors can result in ineffective buddy assignment, particularly where the HR personnel or another employee may not have the complete picture or knowledge of the personalities involved, which can negatively affect productivity and human resource management of the organization.

Different from current onboarding solutions, this specification introduces automatic and machine learning-based leveraging of psychometric profiles to make intelligent and efficient buddy recommendation. For example, employee data is augmented with psychometric profile, and is used to train and run a self-learning ML model for personalized buddy recommendation. Feedback from the new employee and performance information related to the new employee after onboarding can be used to retrain and enhance the self-learning ML model to improve buddy recommendation effectiveness. As a result, new employee's onboarding experience can be improved. In addition, the onboarding buddy recommendation process can make automatic buddy assignments based on buddy efficiency (e.g., how efficient or successful the buddy relationship may be) predicted by the self-learning ML model. In doing so, efficient buddy assignment can be achieved, and the onboarding buddy recommendation process can provide buddy recommendations for a large number of new employees at the same time. Consistent use and learning of the assignments over the whole of an enterprise can consistently improve the onboarding of entire groups of new employees, creating a stronger and more collegial working environment. Similar technologies can be used in any interpersonal solution, including volunteer opportunities, student or mentor-related assignments in education, and any other suitable implementation.

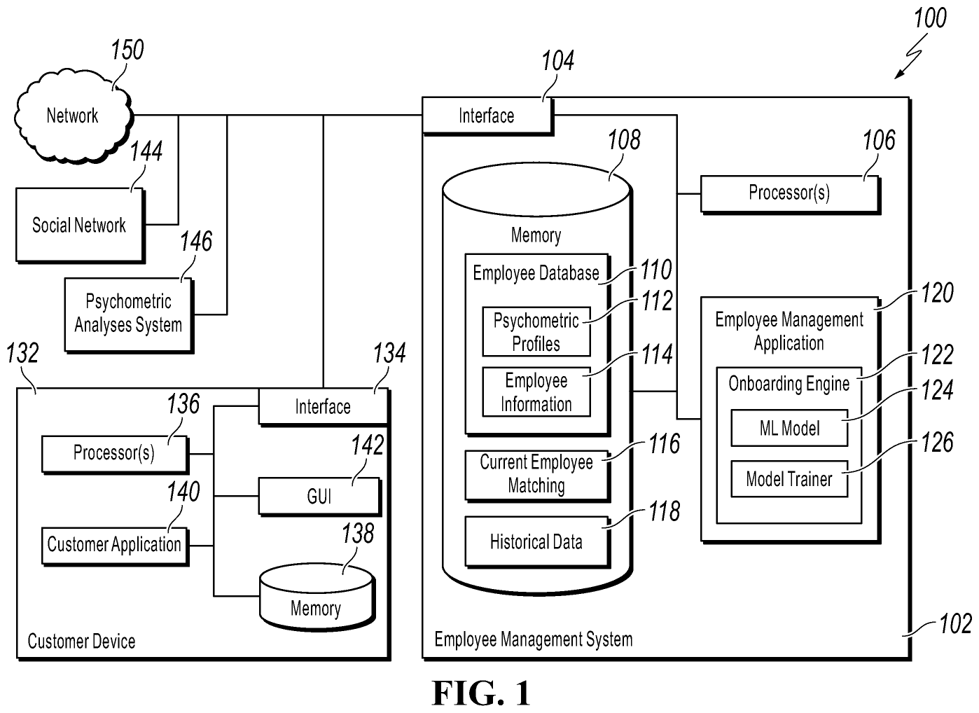

Turning to the illustrated embodiment, FIG. 1 is a block diagram illustrating an example system 100 for humanizing the onboarding buddy recommendation process. Specifically, the illustrated system 100 includes or is communicably coupled with an employee management system 102, a customer device 132, a social network 144, a psychometric analyses system 146, and a network 150. Although shown separately, in some implementations, functionality of two or more systems or servers may be provided by a single system or server. In some implementations, the functionality of one illustrated system, server, or component may be provided by multiple systems, servers, or components, respectively.

As used in the present disclosure, the term “computer” is intended to encompass any suitable processing device. For example, although FIG. 1 illustrates a single employee management system 102 and a single customer device 132, the system 100 can be implemented using a single, stand-alone computing device, two or more servers, or two or more customer device. Indeed, the employee management system 102 may be any computer or processing device such as, a blade server, general-purpose personal computer (PC), Mac®, workstation, UNIX-based workstation, or any other suitable device. In other words, the present disclosure contemplates computers other than general-purpose computers, as well as computers without conventional operating systems. Further, the employee management system 102 and the customer device 132 may each be adapted to execute any operating system, including Linux, UNIX, Windows, Mac OS®, Java™, Android™, iOS, or any other suitable operating system. According to one implementation, the employee management system 102 may also include or be communicably coupled with a communication server, an e-mail server, a web server, a caching server, a streaming data server, and/or other suitable servers or computers.

Organizations, such as small and midsize enterprises (SMEs), large enterprises, or other types of organizations, can use the employee management system 102 for employee management. In general, employee management system 102 may be any suitable computing server or system executing applications related to requests for performing employee management including, for example, onboarding buddy recommendation. The employee management system 102 is described herein in terms of responding to requests for performing employee management from users at customer device 132 and other clients, as well as other systems communicably coupled to network 150 or directly connected to the employee management system 102. However, the employee management system 102 may, in some implementations, be a part of a larger system providing additional functionality. For example, employee management system 102 may be part of an enterprise business application or application suite providing one or more of enterprise relationship management, data management systems, customer relationship management, and others. In one example, employee management system 102 may receive a request to provide a buddy recommendation for a new employee, obtain a psychometric profile of the new employee, access psychometric profiles of current employees from a database, process the psychometric profile of the new employee and the psychometric profiles of the current employees through a self-learning ML model to generate a list of recommended buddies, and respond to the request with a person in the list as a recommended buddy for the new employee. The self-learning ML model can be retrained and enhanced with feedback from and performance information related to the new employee after onboarding to improve buddy recommendation effectiveness. In some implementations, the employee management system 102 may be associated with a particular uniform resource locator (URL) for web-based applications. The particular URL can trigger execution of multiple components and systems.

As illustrated, employee management system 102 includes an interface 104, one or more processors 106, memory 108, and an employee management application 120. In general, the employee management system 102 is a simplified representation of one or more systems and/or servers that provide the described functionality, and is not meant to be limiting, but rather an example of the systems possible.

The interface 104 is used by the employee management system 102 for communicating with other systems in a distributed environment—including within the system 100—connected to the network 150 (e.g., customer device 132, social network 144, psychometric analyses system 146, and other systems communicably coupled to the network 150). Generally, the interface 104 may comprise logic encoded in software and/or hardware in a suitable combination and operable to communicate with the network 150. More specifically, the interface 104 may comprise software supporting one or more communication protocols associated with communications, such that the network 150 or interface's hardware is operable to communicate physical signals within and outside of the illustrated system 100.

Network 150 facilitates wireless or wireline communications between the components of the system 100 (e.g., between employee management system 102 and customer device 132 and among others), as well as with any other local or remote computer, such as additional clients, servers, or other devices communicably coupled to network 150, including those not illustrated in FIG. 1. In the illustrated system, the network 150 is depicted as a single network, but may be comprised of more than one network without departing from the scope of this disclosure, so long as at least a portion of the network 150 may facilitate communications between senders and recipients. In some instances, one or more of the illustrated components may be included within network 150 as one or more cloud-based services or operations. For example, the employee management system 102 may be a cloud-based service. The network 150 may be all or a portion of an enterprise or secured network, while in another instance, at least a portion of the network 150 may represent a connection to the Internet. In some instances, a portion of the network 150 may be a virtual private network (VPN). Further, all or a portion of the network 150 can comprise either a wireline or wireless link. Example wireless links may include 802.11ac/ad/af/a/b/g/n, 802.20, WiMax, LTE, and/or any other appropriate wireless link. In other words, the network 150 encompasses any internal or external network, networks, sub-network, or combination thereof operable to facilitate communications between various computing components inside and outside the illustrated system 100. The network 150 may communicate, for example, Internet Protocol (IP) packets, Frame Relay frames, Asynchronous Transfer Mode (ATM) cells, voice, video, data, and other suitable information between network addresses. The network 150 may also include one or more local area networks (LANs), radio access networks (RANs), metropolitan area networks (MANs), wide area networks (WANs), all or a portion of the Internet, and/or any other communication system or systems at one or more locations.

As illustrated in FIG. 1, the employee management system 102 includes one or more processors 106. Each processor 106 may include a central processing unit (CPU), an application-specific integrated circuit (ASIC), a field-programmable gate array (FPGA), or another suitable component. Generally, each processor 106 executes instructions and manipulates data to perform the operations of the employee management system 102. Specifically, each processor 106 executes the algorithms and operations described in the illustrated figures, including the operations performing the functionality associated with the employee management system 102 generally, as well as the various software modules (e.g., the employee management application 120), including the functionality for sending communications to and receiving transmissions from customer device 132.

The employee management system 102 also includes an employee management application 120. The employee management application 120 provides an onboarding buddy recommendation method using an onboarding engine 122. In operation, the employee management application 120 trains a ML model using historical buddy assignment data, provides buddy recommendation for a new employee based on the ML model, and retrains the ML model using feedback for/from and performance information related to the new employee after onboarding. Operations of the employee management application 120 are executed by the one or more processors 106. In some implementations, the employee management application 120 may be a software program, or set of software programs, executing on the employee management system 102. In various alternative implementations, the employee management application 120 may also be an external component from the employee management system 102 and may communicate with the employee management system 102 over a network (e.g., network 150).

As shown, the employee management application 120 includes the onboarding engine 122. The onboarding engine 122 represents an onboarding buddy recommendation service that automatically recommends or assigns an onboarding buddy for a new employee. In operation, the onboarding engine 122 may obtain psychometric profile of the new employee (e.g., through analyzing one or more social media accounts of the new employee from social network 144, or using psychometric analyses system 146), obtain psychometric profiles of current employees in the memory 108 (e.g., psychometric profiles 112), and provide buddy recommendations by processing the psychometric profile of the new employee and the psychometric profiles of the current employees through the ML model (e.g., ML model 124). For example, posts, likes, stories, and other available information on one or more social media accounts of the new employee (such as Facebook, LinkedIn, and Twitter accounts) can be accessed and analyzed to generate the psychometric profile or a portion of the psychometric profile of the new employee. The psychometric analyses system 146 can be a psychological test website that calculates a psychometric profile of a person after the person completes a psychological test (such as the Big Five personality test) on the website. The onboarding engine 122 may obtain feedback for/from and performance information related to the new employee after onboarding to retrain and enhance the ML model (e.g., model trainer 126). Operations of the onboarding engine 122 are executed by the one or more processors 106. In some implementations, the onboarding engine 122 may be a software program, or set of software programs, executing on the employee management system 102. In various alternative implementations, the onboarding engine 122 may also be an external component from the employee management system 102 and may communicate with the employee management system 102 over a network (e.g., network 150).

The onboarding engine 122 includes the ML model 124. The ML model 124 can be trained using historical data 118 corresponding to historical buddy assignment data. After the ML model 124 is trained, the onboarding engine 122 can use the ML model 124 to generate a buddy prediction for a new employee.

The onboarding engine 122 also includes the model trainer 126. The model trainer 126 can retrain and enhance the ML model 124 to improve buddy recommendation effectiveness. For example, feedback about the buddy assignment, and/or performance information related to the new employee after onboarding can be used to retrain and enhance the ML model 124.

Regardless of the particular implementation, “software” includes computer-readable instructions, firmware, wired and/or programmed hardware, or any combination thereof on a tangible medium (transitory or non-transitory, as appropriate) operable when executed to perform at least one of the processes and operations described herein. In fact, each software component may be fully or partially written or described in any appropriate computer language, including C, C++, JavaScript, JAVA™, VISUAL BASIC, assembler, Perl®, any suitable version of 4GL, as well as others. While portions of the software elements illustrated in FIG. 1 are shown as individual modules that implement the various features and functionality through various objects, methods, or other processes, the software may instead include a number of sub-modules, third-party services, components, libraries, and such, as appropriate. Conversely, the features and functionality of various components can be combined into single components, as appropriate.

As illustrated, employee management system 102 includes memory 108. In some implementations, the employee management system 102 includes multiple memories. The memory 108 may include any memory or database module and may take the form of volatile or non-volatile memory including, without limitation, magnetic media, optical media, random access memory (RAM), read-only memory (ROM), removable media, or any other suitable local or remote memory component. The memory 108 may store various objects or data, including financial and/or business data, application information including URLs and settings, user information, behavior and access rules, administrative settings, password information, caches, backup data, repositories storing business and/or dynamic information, and any other appropriate information including any parameters, variables, algorithms, instructions, rules, constraints, or references thereto associated with the purposes of the employee management system 102. Additionally, the memory 108 may store any other appropriate data, such as VPN applications, firmware logs and policies, firewall policies, a security or access log, print or other reporting files, as well as others. For example, illustrated memory 108 includes employee database 110, current employee matching 116, and historical data 118.

The employee database 110 stores psychometric profiles 112 and employee information 114. For example, the psychometric profiles 112 can include scores quantifying a core set of personality traits of current employee (such as the Big Five personality traits). The employee information 114 can include one or more of name, age, gender, race, education level, location, position, team, tenure, and salary of current employee. The current employee matching 116 stores ongoing buddy assignments. For example, when a current employee is assigned as a buddy to a new employee, this buddy assignment is stored in the current employee matching 116. When the buddy assignment is ended, it can be removed from the current employee matching 116. The historical data 118 stores historical buddy assignment data. For example, information of previous buddy assignments can be collected for a predefined period (such as two years) and stored in the historical data 118. Information of previous buddy assignments can include one or more of psychometric profiles, feedbacks about buddy assignments, and performance information related to new employees after onboarding.

Customer device 132 may be any computing device operable to connect to or communicate with employee management system 102, social network 144, psychometric analyses system 146, other clients (not illustrated), or other components via network 150, as well as with the network 150 itself, using a wireline or wireless connection, and can include a desktop computer, a mobile device, a tablet, a server, or any other suitable computer device. In general, customer device 132 comprises an electronic computer device operable to receive, transmit, process, and store any appropriate data associated with the system 100 of FIG. 1.

As illustrated, customer device 132 includes an interface 134, one or more processors 136, a graphical user interface (GUI) 142, a customer application 140, and memory 138. Interface 134 and one or more processors 136 may be similar to, or different than, interface 104 and one or more processors 106 described with regard to employee management system 102. In general, each processor 136 executes instructions and manipulates data to perform the operations of the customer device 132. Specifically, each processor 136 can execute some or all of the algorithms and operations described in the illustrated figures, including the operations performing the functionality associated with the customer application 140 and the other components of customer device 132. Similarly, interface 134 provides the customer device 132 with the ability to communicate with other systems in a distributed environment—including within the system 100—connected to the network 150.

The customer device 132 includes or presents the GUI 142. For example, the GUI 142 provides a user interface between a user and the customer application 140. In operation, the user uses the GUI 142 to input data about a new employee to whom the user wants to assign an onboarding buddy. For example, the GUI 142 may display multiple fields (e.g., name and team to join) for the user to input data associated with the new employee and to make a request to run the onboarding buddy recommendation process for the new employee. In some implementations, the GUI 142 may display multiple buddy candidates recommended by the onboarding buddy recommendation process and to make a request to assign the user selected buddy candidate as the onboarding buddy for the new employee. In some implementations, the GUI 142 may be a software program, or set of software programs, executing on the customer device 132. The GUI 142 may also be an external component from the customer device 132 and may communicate with the customer device 132 over a network (e.g., network 150).

The GUI 142 of the customer device 132 interfaces with at least a portion of the system 100 for any suitable purpose, including generating a visual representation of the customer application 140 and/or other applications. In particular, the GUI 142 may be used to view and navigate various Web pages, or other user interfaces. Generally, the GUI 142 provides the user with an efficient and user-friendly presentation of business data provided by or communicated within the system. The GUI 142 may comprise a plurality of customizable frames or views having interactive fields, pull-down lists, and buttons operated by the user. The GUI 142 contemplates any suitable graphical user interface, such as a combination of a generic web browser, intelligent engine, and command line interface (CLI) that processes information and efficiently presents the results to the user visually.

The customer device 132 can include one or more client applications, including the customer application 140. In general, a client application is any type of application that allows the customer device 132 to request and view content on the respective device. In some implementations, a client application can use parameters, metadata, and other information received at launch to access a particular set of data from the employee management system 102. In some instances, a client application may be an agent or client-side version of the one or more enterprise applications running on an enterprise server (not shown).

Customer device 132 executes the customer application 140. The customer application 140 may operate with or without requests to the employee management system 102—in other words, the customer application 140 may execute its functionality without requiring the employee management system 102 in some instances, such as by accessing data stored locally on the customer device 132. In others, the customer application 140 may be operable to interact with the employee management system 102 by sending requests via network 150 to the employee management system 102 for performing onboarding buddy recommendation process. For example, a user can use the customer application 140 to request buddy recommendation for a new employee. In some implementations, the customer application 140 may be a standalone web browser, while in others, the customer application 140 may be an application with a built-in browser. The customer application 140 can be a web-based application or a standalone application, developed for the particular customer device 132. For example, the customer application 140 can be a native iOS application for iPad, a desktop application for laptops, as well as others.

Memory 138 may be similar to or different from memory 108 of the employee management system 102. In some implementations, the customer device 132 includes multiple memories. In general, memory 138 may store various objects or data, including any parameters, variables, algorithms, instructions, rules, constraints, or references thereto, associated with the purposes of the customer device 132. Additionally, the memory 138 may store any other appropriate data, such as VPN applications, firmware logs and policies, firewall policies, a security or access log, print or other reporting files, as well as others.

The illustrated customer device 132 is intended to encompass any computing device such as a desktop computer, laptop/notebook computer, mobile device, smartphone, personal data assistant (PDA), tablet computing device, one or more processors within these devices, or any other suitable processing device. For example, the customer device 132 may comprise a computer that includes an input device, such as a keypad, touch screen, or other device that can accept user information, and an output device that conveys information associated with the operation of the customer application 140 or the customer device 132 itself, including digital data, visual information, or the GUI 142, as shown with respect to the customer device 132. Further, while illustrated as a client system, customer device 132 may be exchanged with another suitable source for performing onboarding buddy recommendation in other implementations, and is not meant to be limiting.

There may be any number of customer devices 132 associated with, or external to, the system 100. For example, while the illustrated system 100 includes one customer device 132, alternative implementations of the system 100 may include multiple customer devices 132 communicably coupled to the employee management system 102 and/or the network 150, or any other number suitable to the purposes of the system 100. Additionally, there may also be one or more additional customer devices 132 external to the illustrated portion of system 100 that are capable of interacting with the system 100 via the network 150. Further, the term “client”, “client device” and “user” may be used interchangeably as appropriate without departing from the scope of this disclosure. Moreover, while the customer device 132 is described in terms of being used by a single user, this disclosure contemplates that many users may use one computer, or that one user may use multiple computers.

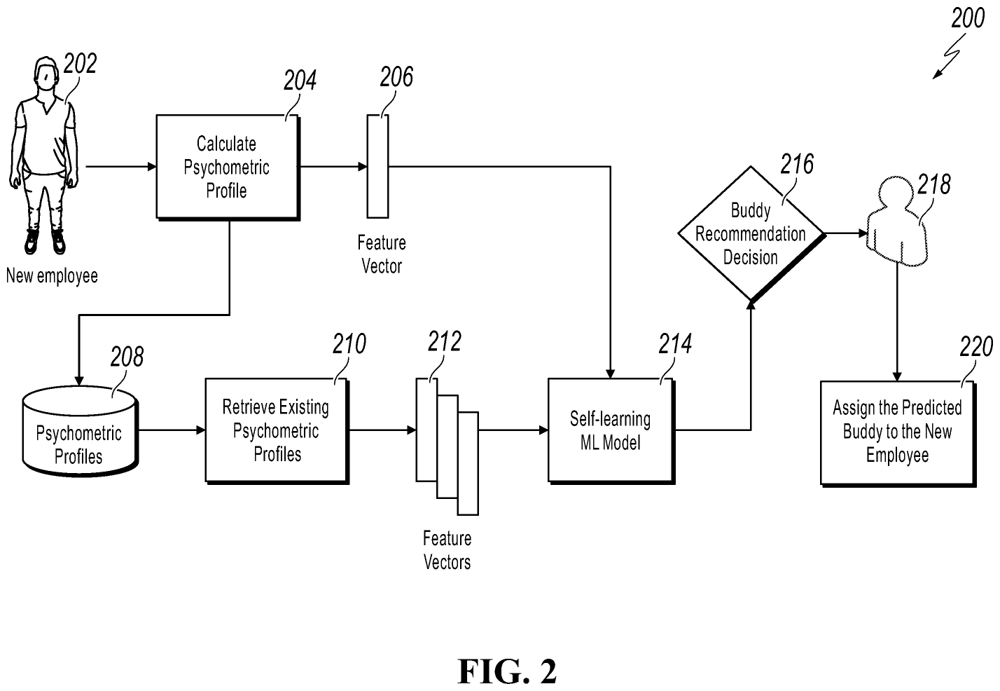

FIG. 2 is a flow diagram of an example process 200 for predicting and assigning an onboarding buddy. Operations of process 200 are described below as being performed by one or more components of the system 100 described above with respect to FIG. 1. For example, the process 200 can be executed by the employee management system 102 of FIG. 1. Operations of the process 200 are described below for illustration purposes only. Operations of the process 200 can be performed by any appropriate device or system, e.g., any appropriate data processing apparatus. Operations of the process 200 can also be implemented as instructions stored on a non-transitory computer readable medium. Execution of the instructions causes one or more data processing apparatus to perform operations of the process 200.

After a new employee joins an organization at 202, an onboarding buddy needs to be assigned to the new employee. For example, the new employee can be a person newly joining the organization, or a current employee transferring to a new team within the organization. The onboarding buddy can be selected from the current employees of the organization, and explain processes, facilities, policies, and teams of the organization to the new employee. In some implementations, the onboarding buddy is assigned to the new employee for a predefined time period (such as a month).

At 204, a psychometric profile of the new employee is calculated. The psychometric profile of the new employee can include a score quantifying a core set of personality traits of the new employee (such as the Big Five personality traits). For example, the core set of personality traits includes openness to experience, conscientiousness, extraversion, agreeableness, and neuroticism. A sample Big Five personality test result of [91, 85, 54, 88, 9], based on a scale from 0 to 100, indicates a high score of 91 on openness to experience (e.g., imaginative and open-minded), a high score of 85 on conscientiousness (e.g., hardworking, reliable, and principled), a middle score of 54 on extraversion (e.g., fairly friendly), a high score of 88 on agreeableness (e.g., compassionate and unselfish), and a low score of 9 on neuroticism (e.g., well balanced, relaxed, and resilient).

The psychometric profile of the new employee can be calculated based on a questionnaire filled out by the new employee, or through analyzing one or more social media accounts of the new employee (such as Facebook and Twitter accounts). In some implementations, the psychometric profile of the new employee can be calculated by a third-party system (such as psychometric analyses system 146 of FIG. 1). In some instances, the organization can periodically (such as monthly) recalculate current employees' psychometric profiles, thereby providing a regularly updated set of results and recommendations.

At 206, a feature vector of the new employee is retrieved based on the psychometric profile of the new employee. For example, the psychometric profile can be the only feature or the primary feature in the feature vector. In some implementations, in addition to psychometric profile, the feature vector can include other information of the new employee. For example, the feature vector can include one or more of team, location, tenure, performance, job satisfaction, employee engagement, education level, and background. In some implementations, different weights can be applied to different features in the feature vector. For example, if the psychometric profile is considered to be the most important feature in the feature vector, weight applied to the psychometric profile is the highest in the feature vector.

At 208, the psychometric profile of the new employee is stored in an employee database. For example, the employee database can be a core HR master database maintaining employee data for employee management. In addition to the psychometric profile, the employee database can store other employee information (such as name, age, gender, race, education level, location, position, team, tenure, and salary).

At 210, psychometric profiles of current employees are retrieved from the employee database. For example, psychometric profiles of all current employees are retrieved when all current employees are available to be assigned as the onboarding buddy. In some implementations, psychometric profiles of some current employees, who are available to be assigned as the onboarding buddy, are retrieved. For example, employees, who are on vacation, currently in a buddy relationship, or in a different team than the new employee, may not be available to be assigned as the onboarding buddy.

At 212, feature vectors of current employees can be retrieved based on the psychometric profiles of the current employees. For example, the psychometric profile can be the only feature or the primary feature in each feature vector. In some implementations, in addition to the psychometric profile, each feature vector can include other information of a current employee. For example, each feature vector can include one or more of team, location, tenure, performance, job satisfaction, employee engagement, education level, and background. In some implementations, features included in feature vectors at 212 may be the same as or similar to features included in feature vector at 206. In some implementations, different weights can be applied to different features in each feature vector. In some implementations, weights applied at 212 may be the same as or similar to weights applied at 206.

At 214, the feature vector of the new employee and feature vectors of the current employees are input into a self-learning ML model to perform buddy recommendations. In some implementations, additional information (such as team to join) can also be input into the self-learning ML model. The output of the self-learning ML model can include one or more potential buddy candidates. For example, each buddy candidate is a current employee in a same team as the new employee, and is associated with a predicted buddy efficiency higher than a predefined threshold (such as 80%). The predicted buddy efficiency can indicate how efficient or successful the buddy relationship may be. In some implementations, the self-learning ML model can calculate buddy efficiencies between any current employee and the new employee, rank the current employees based on the calculate buddy efficiencies, and generate a list of buddy candidates based on the ranking (such as top three ranked employees). In some instances, where different weighting of feature vector components may be available, two or more lists may be generated, along with an explanation of which weighting caused which list, thereby providing additional information in the result set.

In some implementations, the self-learning ML model has been trained with historical buddy assignment data before performing the buddy recommendation. For example, information of previous buddy assignments can be collected and used as training data for the self-learning ML model. The training data can be collected for a predefined period (such as two years). For each previous buddy assignment, the training data can include one or more of psychometric profiles of the buddy and the new employee, feedbacks about the buddy assignment from the new employee, the buddy, and/or managers or others involved in the buddy evaluation, and performance information related to the new employee during and after onboarding. For example, after buddy assignment ends, a survey or a feedback link can be sent to the new employee and/or the buddy, which can be used to measure quality of the buddy assignment. Additionally, employee retention information for different buddy pairings can be used to determine relative lengths of employment post-onboarding for those assigned buddies.

At 216, buddy recommendation decision is made based on the output of the self-learning ML model. For example, if the output of the self-learning ML model includes a list of buddy candidates ranked with buddy efficiencies, the buddy candidate with the highest ranking can be automatically selected as the predicted buddy at 218. In some implementations, the list is provided to a manager or an HR personnel for selection, and the selected buddy candidate can be automatically selected as the predicted buddy at 218. The selection of the manager or the HR personnel can be used to retrain and enhance the self-learning ML model.

At 220, the predicted buddy is assigned as the onboarding buddy to the new employee within the organization. For example, the predicted buddy is assigned to the new employee for a predefined time period (such as a month). In some implementations, the buddy assignment is stored in a database (such as current employee matching 116 of FIG. 1), and is removed from the database when the buddy assignment is ended. In some implementations, the predicted buddy is not automatically assigned to the new employee. Instead, the predicted buddy is provided to a manager or an HR personnel to confirm or update. In some instances, the manager or the HR personnel can identify a change to the weighting for a particular role after the buddy recommendations are provided, and the process 200 can generate new recommendations based on the changed weighting.

In some implementations, the self-learning ML model can be retrained and enhanced with feedbacks about the buddy assignment from the new employee and/or the buddy, and/or performance information related to the new employee after onboarding. For example, after the buddy assignment ends, a survey or a feedback link can be sent to the new employee and/or the buddy to measure quality of the buddy assignment. Performance information related to the new employee (such as tenure, engagement, and satisfaction) after joining the organization for a predefined period (such as a year) can also be used to measure quality of the buddy assignment.

In some implementations, the self-learning ML model can provide automated buddy recommendation for a large number of new employees (such as 1,000). For example, the self-learning ML model can automatically assign a current employee as a buddy to a new employee as described above, remove the assigned current employee from consideration as buddy candidates for other new employees, and repeat the buddy assignment process for each of the other new employees.

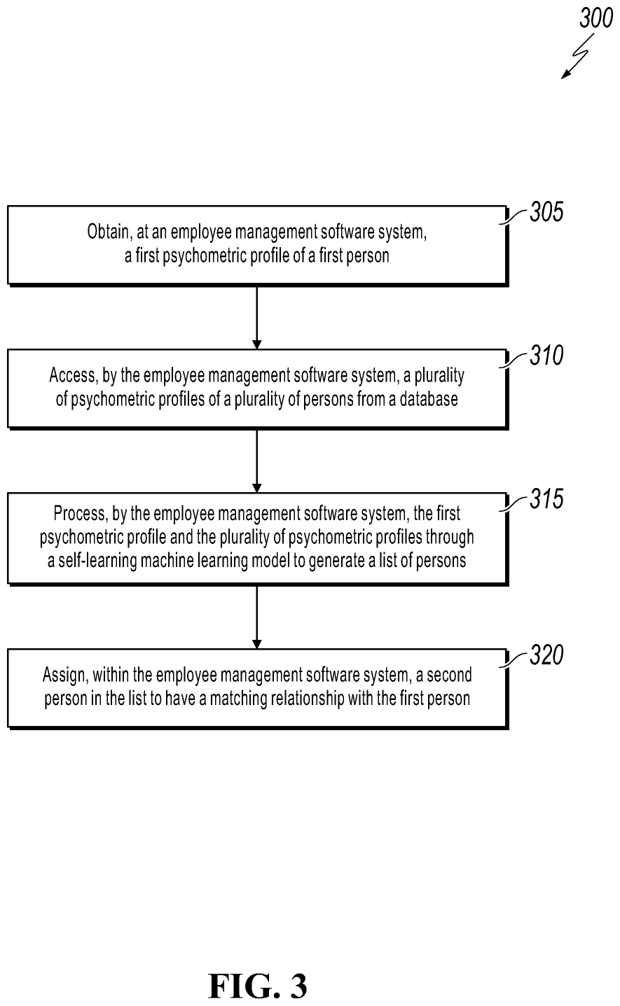

FIG. 3 is a flowchart of an example method for humanizing the onboarding buddy recommendation process. It will be understood that method 300 and related methods may be performed, for example, by any suitable system, environment, software, and hardware, or a combination of systems, environments, software, and hardware, as appropriate. For example, one or more of a client, a server, or other computing device can be used to execute method 300 and related methods and obtain any data from the memory of a client, the server, or the other computing device. In some implementations, the method 300 and related methods are executed by one or more components of the system 100 described above with respect to FIG. 1. For example, the method 300 and related methods can be executed by the employee management system 102 of FIG. 1.

At 305, a first psychometric profile of a first person is obtained at an employee management software system. The first person can be a person newly joining an organization, or a current employee transferring to a new team within the organization. The employee management software system can include an onboarding system platform of the organization for performing onboarding buddy recommendation process.

The first psychometric profile of the first person includes a score quantifying a core set of personality traits of the first person (such as the Big Five personality traits). For example, the core set of personality traits includes openness to experience, conscientiousness, extraversion, agreeableness, and neuroticism. The first psychometric profile of the first person can be calculated based on a questionnaire filled by the first person, or through analyzing one or more social media accounts of the first person (such as Facebook and Twitter accounts). In some implementations, the first psychometric profile of the first person can be calculated using a third-party system (such as a psychological test website). In some implementations, other information (such as team, location, tenure, performance, job satisfaction, employee engagement, education level, and background) of the first person can be extracted from the third-party system.

At 310, a plurality of psychometric profiles of a plurality of persons are accessed by the employee management software system from a database. The plurality of persons can be current employees of the organization. In some implementations, the plurality of persons are in a same team as the first person. The database can be an employee database maintaining employee data for employee management. In addition to psychometric profile, the database can store one or more of name, age, gender, race, education level, location, position, team, tenure, and salary. In some implementations, the obtained first psychometric profile at 305 is stored in the database.

At 315, the first psychometric profile and the plurality of psychometric profiles are processed by the employee management software system through a self-learning ML model to generate a list of persons. In some implementations, additional information (such as team to join) can also be input into the self-learning ML model. The list of persons can include one or more buddy candidates. For example, each buddy candidate is a current employee in a same or related team as the first person, and is associated with a predicted buddy efficiency higher than a predefined threshold. The predicted buddy efficiency can indicate how efficient or successful the buddy relationship may be. In some implementations, the self-learning ML model can calculate buddy efficiencies between anyone of the plurality of persons and the first person, rank the plurality of persons based on the calculate buddy efficiencies, and generate the list of persons based on the ranking (such as top three ranked persons).

In some implementations, processing the first psychometric profile and the plurality of psychometric profiles can include retrieving a first feature vector of the first person from the first psychometric profile, retrieving a plurality of feature vectors of the plurality of persons from the plurality of psychometric profiles, and comparing the first feature vector to the plurality of feature vectors. For example, psychometric profile can be the only feature or the primary feature in a feature vector. In some implementations, in addition to psychometric profile, the feature vector can include other information. For example, the feature vector can include one or more of team, location, tenure, performance, job satisfaction, employee engagement, education level, and background. In some implementations, different weights can be applied to different features in the feature vector. For example, if psychometric profile is considered to be the most important feature in the feature vector, weight applied to psychometric profile is the highest in the feature vector.

At 320, a second person in the list is assigned, within the employee management software system, to have a matching relationship with the first person. For example, the matching relationship can include a buddy relationship or a mentorship, and the second person is assigned as a buddy or a mentor to the first person for a predefined time period (such as a month). In some implementations, the matching relationship is stored in the database (such as current employee matching 116 of FIG. 1), and is removed from the database when the matching relationship is ended.

In some implementations, the employee management software system can automatically select a person in the list with a relatively highest predicted buddy efficiency as the second person, and assign to the first person. In some implementations, the list is provided to a manager or an HR personnel for selection, and the selected person can be automatically selected as the second person, and assigned to the first person. The selection of the manager or the HR personnel can be used to retrain and enhance the self-learning ML model.

In some implementations, before processing the first psychometric profile and the plurality of psychometric profiles, the self-learning ML model is trained with historical data. For example, information of previous matching relationships can be collected and used as training data for the self-learning ML model. The training data can be collected for a predefined period (such as two years). For each previous matching relationship, the training data can include one or more of psychometric profiles, feedbacks about the matching relationship, and performance information related to the first person after onboarding. For example, after the matching relationship ends, a survey or a feedback link can be sent to the first person and/or the second person, which can be used to measure quality of the matching relationship.

In some implementations, the self-learning ML model can be retrained and enhanced with feedbacks about the matching relationship, and/or performance information related to the first person after onboarding. For example, after the matching relationship ends, a survey or a feedback link can be sent to the first person and/or the second person to measure quality of the matching relationship. Performance information related to the first person (such as tenure, engagement, and satisfaction) after joining the organization for a predefined period (such as a year) can also be used to measure quality of the matching relationship.

In some implementations, the self-learning ML model can automatically establish matching relationships for a large number of new employees (such as 1,000). For example, the self-learning ML model can automatically assign a current employee to have a matching relationship with a new employee, remove the assigned current employee from consideration to have matching relationships with other new employees, and repeat the process for each of the other new employees. In other instances, employees may be buddies to two or more new employees. However, the fact that an employee is already assigned a new employee buddy may cause their relative efficiency score to be reduced, as having multiple buddies may lessen the effectiveness of the current and additional buddy assignments. However, those employees may still be considered when their personality and the new employee's are such a match that the potential downside is overcome.

The preceding figures and accompanying description illustrate example processes and computer-implementable techniques. But system 100 (or its software or other components) contemplates using, implementing, or executing any suitable technique for performing these and other tasks. It will be understood that these processes are for illustration purposes only and that the described or similar techniques may be performed at any appropriate time, including concurrently, individually, or in combination. In addition, many of the operations in these processes may take place simultaneously, concurrently, and/or in different orders than as shown. Moreover, system 100 may use processes with additional operations, fewer operations, and/or different operations, so long as the methods remain appropriate.

In other words, although this disclosure has been described in terms of certain embodiments and generally associated methods, alterations and permutations of these embodiments and methods will be apparent to those skilled in the art. Accordingly, the above description of example embodiments does not define or constrain this disclosure. Other changes, substitutions, and alterations are also possible without departing from the spirit and scope of this disclosure.

What is claimed is:

1. A computer-implemented method comprising:
obtaining, at an employee management software system, a first psychometric profile of a first person;
accessing, by the employee management software system, a plurality of psychometric profiles of a plurality of persons from a database;
processing, by the employee management software system, the first psychometric profile and the plurality of psychometric profiles through a self-learning machine learning model to generate a list of persons; and
assigning, within the employee management software system, a second person in the list to have a matching relationship with the first person.

2. The computer-implemented method of claim 1, wherein processing the first psychometric profile and the plurality of psychometric profiles through the self-learning machine learning model comprises:
retrieving a first feature vector of the first person from the first psychometric profile;
retrieving a plurality of feature vectors of the plurality of persons from the plurality of psychometric profiles; and
comparing the first feature vector to the plurality of feature vectors.

3. The computer-implemented method of claim 1, wherein, before processing the first psychometric profile and the plurality of psychometric profiles, the self-learning machine learning model is trained with historical data.

4. The computer-implemented method of claim 1, wherein the list of persons are derived from the plurality of persons, each person in the list is associated with a predicted efficiency of a matching relationship with the first person, and the second person has a relatively highest predicted efficiency among the list of persons.

5. The computer-implemented method of claim 1, wherein the first psychometric profile of the first person includes a score quantifying a core set of personality traits of the first person.

6. The computer-implemented method of claim 5, wherein the core set of personality traits includes openness to experience, conscientiousness, extraversion, agreeableness, and neuroticism.

7. The computer-implemented method of claim 5, wherein the core set of personality traits is obtained through a questionnaire or through analyzing one or more social media accounts of the first person.

8. The computer-implemented method of claim 1, wherein the first person is a new employee of an organization, the plurality of persons are current employees of the organization, and the employee management software system includes an onboarding system platform of the organization.

9. The computer-implemented method of claim 1, further comprising storing the first psychometric profile in the database.

10. The computer-implemented method of claim 1, wherein the matching relationship includes a buddy relationship or a mentorship.

11. A system comprising:
one or more computers; and
a computer-readable medium coupled to the one or more computers having instructions stored thereon which, when executed by the one or more computers, cause the one or more computers to perform operations comprising:
obtaining a first psychometric profile of a first person;
accessing a plurality of psychometric profiles of a plurality of persons from a database;
processing the first psychometric profile and the plurality of psychometric profiles through a self-learning machine learning model to generate a list of persons; and
assigning a second person in the list to have a matching relationship with the first person.

12. The system of claim 11, wherein processing the first psychometric profile and the plurality of psychometric profiles through the self-learning machine learning model comprises:
retrieving a first feature vector of the first person from the first psychometric profile;
retrieving a plurality of feature vectors of the plurality of persons from the plurality of psychometric profiles; and
comparing the first feature vector to the plurality of feature vectors.

13. The system of claim 11, wherein, before processing the first psychometric profile and the plurality of psychometric profiles, the self-learning machine learning model is trained with historical data.

14. The system of claim 11, wherein the list of persons are derived from the plurality of persons, each person in the list is associated with a predicted efficiency of a matching relationship with the first person, and the second person has a relatively highest predicted efficiency among the list of persons.

15. The system of claim 11, wherein the first psychometric profile of the first person includes a score quantifying a core set of personality traits of the first person.

16. The system of claim 15, wherein the core set of personality traits includes openness to experience, conscientiousness, extraversion, agreeableness, and neuroticism.

17. The system of claim 15, wherein the core set of personality traits is obtained through a questionnaire or through analyzing one or more social media accounts of the first person.

18. The system of claim 11, wherein the first person is a new employee of an organization, the plurality of persons are current employees of the organization, and the system includes an onboarding system platform of the organization.

19. The system of claim 11, the operations further comprising storing the first psychometric profile in the database.

20. A computer program product encoded on a non-transitory storage medium, the product comprising non-transitory, computer readable instructions for causing one or more processors to perform operations comprising:
obtaining, at an employee management software system, a first psychometric profile of a first person;
accessing, by the employee management software system, a plurality of psychometric profiles of a plurality of persons from a database;
processing, by the employee management software system, the first psychometric profile and the plurality of psychometric profiles through a self-learning machine learning model to generate a list of persons; and
assigning, within the employee management software system, a second person in the list to have a matching relationship with the first person.
